(19) United States
(12) Patent Application Publication
(10) Pub. No.: US 2026/0301957 A1
(43) Pub. Date: Oct. 1, 2026

(54) Computer-Implemented Method for Providing Recommendation Information for at Least One Magnetic Resonance Protocol for a Magnetic Resonance Examination

(71) Applicant: Siemens Healthineers AG, Forchheim (DE)

(72) Inventors: Fabian Wagner, Erlangen (DE); Tobias Würfl, Erlangen (DE); Julian Wohlers, Erlangen (DE); Rainer Schneider, Höchstadt (DE)

(73) Assignee: Siemens Healthineers AG, Forchheim (DE)

(21) Appl. No.: 19/630,676

(22) Filed: Mar. 27, 2026

(30) Foreign Application Priority Data
Mar. 28, 2025 (EP) 25167049.3

(51) Int. Cl. G16H 50/20; G16H 30/00; G16H 40/63; G16H 50/70

(52) U.S. Cl. CPC G16H 50/20; G16H 30/00; G16H 40/63; G16H 50/70

(57) ABSTRACT

A computer-implemented method for providing recommendation information for at least one magnetic resonance protocol for a magnetic resonance examination on a patient is described. The method may include: providing at least one item of examination-relevant patient information and/or at least one item of examination-relevant operator information; providing protocol information on different magnetic resonance protocols from a totality of available magnetic resonance protocols; determining, by applying a trained language model to the patient information and/or operator information, a patient feature vector; determining, by applying the trained language model to the protocol information, a protocol feature vector for each magnetic resonance protocol; applying a similarity algorithm to the patient feature vector and the different protocol feature vectors to determine recommendation information comprising at least one magnetic resonance protocol having a maximum match with the patient feature vector; and providing the recommendation information.

### CROSS REFERENCE TO RELATED APPLICATIONS

This patent application claims priority to, and the benefit of, European Patent Application No. 25167049.3, filed Mar. 28, 2025, which is incorporated herein by reference in its entirety.

### BACKGROUND

To clarify clinical and/or diagnostic issues in patients, a magnetic resonance examination is often carried out on the patient. For this purpose, medical operating personnel create and/or select a magnetic resonance protocol, which specifies an examination procedure in the magnetic resonance examination. Such a magnetic resonance protocol preferably comprises a plurality of magnetic resonance sequences, which are executed one after the other in a defined order.

When selecting a magnetic resonance protocol, medical operating personnel are intended to take into account the clinical and/or diagnostic issue to be clarified and/or an anatomy to be mapped and/or further patient data. In addition, there is the option when selecting a magnetic resonance protocol to select a particular scan strategy, for example a particularly fast measurement or a measurement that has a particularly high image resolution or even a particularly quiet measurement. Moreover, a selected scan strategy can be differentiated further, for instance whether a contrast agent is intended to be administered to the patient during the measurement or whether the measurement must take account of an implant in the patient, etc.

Currently, planning a magnetic resonance examination, and thus selecting a magnetic resonance protocol for the magnetic resonance examination, is carried out by medical operating personnel, for instance a radiologist or a doctor. This is a very time-consuming process, however, requiring a large amount of experience on the part of the medical operating personnel. For example, selecting a magnetic resonance protocol requires precise identification of the magnetic resonance protocols from a protocol tree, which is stored and/or configured in a protocol database of a magnetic resonance apparatus.

Selecting the magnetic resonance protocol is mostly carried out by an assistant, who in turn should have precise knowledge of the protocol tree and of the designation and/or working principle of the individual magnetic resonance protocols in the protocol tree. In addition, it is also useful here to take into account examination-relevant patient information, for instance if a patient is likely to move. This can lead to inconsistencies and non-standardized protocol selections, however. In addition, if medical operating personnel are inexperienced, an incorrect protocol may be assigned, which can lead to unusable measurement data and/or to time-consuming repetition of the magnetic resonance examination.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 shows a flow diagram of a method according to the disclosure for providing recommendation information for at least one magnetic resonance protocol for a magnetic resonance examination;

FIG. 2 shows a data flow diagram of the method for providing recommendation information for at least one magnetic resonance protocol for a magnetic resonance examination;

FIG. 3 shows a schematic representation of a computer for regulating and/or controlling the method; and

FIG. 4 shows a system with the computer.

The exemplary embodiments of the present disclosure will be described with reference to the accompanying drawings. Elements, features and components that are identical, functionally identical and have the same effect are—insofar as is not stated otherwise—respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software or a combination of hardware and software.

The present disclosure relates to a computer-implemented method for providing recommendation information for at least one magnetic resonance protocol for a magnetic resonance examination on a patient. The disclosure also relates to a computer for regulating and/or controlling a method for providing recommendation information for at least one magnetic resonance protocol for a magnetic resonance examination on a patient. In addition, the disclosure relates to a system for providing recommendation information for at least one magnetic resonance protocol for a magnetic resonance examination on a patient. A further aspect of the disclosure comprises a computer program product with program elements for executing the method for providing recommendation information for at least one magnetic resonance protocol for a magnetic resonance examination. An object of the present disclosure is to provide reliable and rapid assistance to medical operating personnel in selecting a magnetic resonance protocol.

The disclosure is based on a computer-implemented method for providing recommendation information for at least one magnetic resonance protocol for a magnetic resonance examination on a patient, comprising the following method steps:
providing at least one item of examination-relevant patient information and/or at least one item of examination-relevant operator information;
providing protocol information on different magnetic resonance protocols from a totality of available magnetic resonance protocols;
applying a trained language model to the at least one item of examination-relevant patient information and/or the at least one item of examination-relevant operator information, wherein, by means of the trained language model, a patient feature vector is determined and provided;
applying a trained language model to the protocol information of the different magnetic resonance protocols, wherein, by means of the trained language model, a protocol feature vector is determined and provided for each magnetic resonance protocol of the different magnetic resonance protocols;
applying a similarity algorithm to the patient feature vector and the different protocol feature vectors and determining recommendation information comprising at least one magnetic resonance protocol, wherein the protocol feature vector of the at least one magnetic resonance protocol in the recommendation information has a maximum match with the patient feature vector; and
providing the recommendation information.

The method for providing recommendation information for at least one magnetic resonance protocol for a magnetic resonance examination on a patient may be configured to assist medical operating personnel, for example a doctor and/or radiologist, in selecting a magnetic resonance protocol for an upcoming magnetic resonance examination on a patient. The user receives recommendation information for at least one magnetic resonance protocol, wherein the selected or recommended at least one magnetic resonance protocol has a best match with the requirements, for example with patient information and/or internal work instructions. The method may be executed in an automated manner. In this regard, medical operating personnel must merely start the planning of the magnetic resonance examination on the patient, and then automatically receive the recommendation information provided.

The magnetic resonance protocol may comprise a plurality of magnetic resonance sequences, wherein the magnetic resonance protocol sets a defined order of execution of the plurality of magnetic resonance sequences. If intermediate steps are needed for the upcoming magnetic resonance examination, for instance administering contrast agent and/or repositioning the patient, etc., the timing of the intermediate steps between the individual magnetic resonance sequences is also set by the magnetic resonance protocol. The individual magnetic resonance sequences may be tailored to a clinical and/or diagnostic issue to be clarified by the magnetic resonance examination on the patient.

The individual magnetic resonance sequences may comprise a plurality of radiofrequency pulses and/or a plurality of gradient pulses, wherein the plurality of radiofrequency pulses and/or the plurality of gradient pulses are emitted in a defined order in time, such as radiated into a field of view (FoV) of a magnetic resonance apparatus. For this purpose, the magnetic resonance may include a radiofrequency antenna unit and a gradient coil unit, which are configured to emit the radiofrequency pulses and the gradient pulses.

The examination-relevant patient information may be stored digitally in at least one database and/or at least one memory unit, for example in a digital patient database and/or a radiology information system (RIS). The examination-relevant patient information comprises a current specific clinical and/or diagnostic issue that is the reason for the upcoming magnetic resonance examination on the patient. In an exemplary embodiment, the examination-relevant patient information comprises all the information stored on the patient and/or data stored, for example, in a patient database and/or an RIS database.

The at least one item of examination-relevant operator information may comprise examination-relevant information of an operator, where an operator can be a doctor, a hospital, a radiology center, etc. The examination-relevant operator information can comprise, for example, a local and/or owner-dependent regulation and/or information relating to the magnetic resonance examination.

The providing of the at least one item of examination-relevant patient information and/or the at least one item of examination-relevant operator information may be performed by means of a provision unit. The provision unit can be comprised by the magnetic resonance apparatus. In addition, the provision unit can also be comprised by the database in which is stored the at least one item of examination-relevant patient information and/or the at least one item of examination-relevant operator information. In addition, the provision unit can also be comprised by a computer, which computer can be used for planning the magnetic resonance examination of the patient by the medical operating personnel, and is designed in particular to control the method to provide recommendation information for at least one magnetic resonance protocol for a magnetic resonance examination on a patient.

The providing of the at least one item of examination-relevant patient information and/or the at least one item of examination-relevant operator information can also comprise generating from the at least one item of examination-relevant patient information and/or the at least one item of examination-relevant operator information a text string, which is provided for analysis or revising by a trained language model. The generating of the text string from the at least one item of examination-relevant patient information and/or the at least one item of examination-relevant operator information may be performed by the computer.

The protocol information for a magnetic resonance protocol can comprise, in the simplest case, just the name of the protocol. In an exemplary embodiment, the protocol information for a magnetic resonance protocol comprises additional information on the magnetic resonance protocol, such as which magnetic resonance sequences are used in the magnetic resonance protocol and/or what properties this magnetic resonance protocol comprises and/or what settings this magnetic resonance protocol has, etc.

The totality of the available magnetic resonance protocols may comprise all the magnetic resonance protocols that are stored locally and/or on site for use with the selected and/or available magnetic resonance apparatus in a local database. The totality of the available magnetic resonance protocols can be stored and/or sorted in a protocol tree. For example, the individual magnetic resonance protocols are sorted in a protocol tree according to the body regions under examination in patients. In addition, further sorting of the magnetic resonance protocols that is considered useful by a person skilled in the art is also readily conceivable.

The providing of protocol information on different magnetic resonance protocols from the totality of available magnetic resonance protocols can be restricted to those different magnetic resonance protocols that cover the body region relevant to the upcoming magnetic resonance examination on the patient. In an exemplary embodiment, the totality of the available magnetic resonance protocols is divided into different protocol groups, with each different protocol group being assigned to a defined body region. A protocol group may include all the magnetic resonance protocols that are designed for a magnetic resonance examination of this defined body region. For example, for a clinical and/or diagnostic issue relating to the head region of the patient can be provided only magnetic resonance protocols that are assigned to the “head” protocol group and designed and/or developed for head examinations. In another example, if the patient has a knee problem, can be provided only magnetic resonance protocols that are assigned to the “knee” protocol group and designed and/or developed for knee examinations.

The providing of the protocol information on different magnetic resonance protocols from a totality of available magnetic resonance protocols may be performed by means of a provision unit. The provision unit can be comprised by the magnetic resonance apparatus. In addition, the provision unit can also be comprised by the database in which is stored the totality of available magnetic resonance protocols. In addition, the provision unit can also be comprised by a computer, which computer can be used for planning the magnetic resonance examination of the patient by the medical operating personnel, and is designed in particular to control the method to provide recommendation information for at least one magnetic resonance protocol for a magnetic resonance examination on a patient.

The providing of the protocol information on different magnetic resonance protocols from the totality of available magnetic resonance protocols can also comprise generating different text strings, with each text string being associated with precisely one magnetic resonance protocol. The different text strings can then be provided for analysis or revising by the trained language model. The generating of the text strings for the protocol information of the different magnetic resonance protocols may be performed by the computer.

The language model (LM) is a mathematical model, which models the series of elements in a sequence, for instance of letters and/or words. The language model can be configured such that it recognizes and/or understands natural speech and in particular individual elements such as words in inputs that contain natural language, and transcribes the elements into a text output. The natural speech processing algorithm may be based on a trained function or function trained by machine learning. Alternatively, the algorithm for processing natural speech can be rule-based.

Particularly advantageously, the trained language model comprises a trained large language model (LLM), in particular an embedded LLM. The trained LLM can represent a computer linguistics probabilistic model, which has learned statistical word-order and sentence-order relationships from a large number of text documents through a computationally intensive training process. In particular, the probabilities of the trained LLM are calculated by artificial neural networks.

The trained LLM may be designed and/or trained to analyze the examination-relevant patient information and/or the examination-relevant operator information, and to generate a patient feature vector therefrom. In particular, the trained LLM is designed and/or trained to analyze the provided text string generated from the examination-relevant patient information and/or the examination-relevant operator information, and to generate a patient feature vector therefrom. The patient feature vector may vectorially summarize the parameterizable properties of a pattern, in particular of the examination-relevant patient information and/or of the examination-relevant operator information in the text string. Different features that are characteristic of the examination-relevant patient information and/or the examination-relevant operator information can map different dimensions of the patient feature vector. The patient feature vector comprises a sequence of numbers that comprise and/or represent the characteristic features of the examination-relevant patient information and/or the examination-relevant operator information.

Furthermore, the trained LLM may be designed and/or trained to analyze the protocol information for the different magnetic resonance protocols, and to generate therefrom a protocol feature vector for each magnetic resonance protocol of the different magnetic resonance protocols. In particular, the trained LLM is designed and/or trained to analyze the provided text strings generated from the protocol information, and to generate therefrom a protocol feature vector for each text string. The protocol feature vector may vectorially summarize the parameterizable properties of a pattern, in particular of a text string of protocol information of a magnetic resonance protocol. Different features that are characteristic of the magnetic resonance protocol can map different dimensions of the protocol feature vector. The protocol feature vector comprises a sequence of numbers that represent the characteristic features of the magnetic resonance protocol and/or of the protocol information of the magnetic resonance protocol. Thus different protocol feature vectors are determined and provided by the trained LLM for the different magnetic resonance protocols. In particular, one protocol feature vector is generated and provided for each magnetic resonance protocol.

The applying of the trained language model, in particular the trained LLM, to the at least one item of examination-relevant patient information and/or the at least one item of examination-relevant operator information is carried out by a computer, which comprises for this purpose the trained language model, in particular the trained LLM. In addition, the applying of the trained language model, in particular the trained LLM, to the protocol information of the different magnetic resonance protocols is also carried out by means of the computer. The computer has in addition an input interface, which enables access to the at least one item of examination-relevant patient information and/or the at least one item of examination-relevant operator information. The input interface of the computer additionally also enables access to the protocol information of the different magnetic resonance protocols.

In an exemplary embodiment, the trained language model, in particular the trained LLM, is used both for determining and/or generating the patient feature vector and for determining and/or generating the protocol feature vectors. The patient feature vector and the plurality of protocol feature vectors are provided by the trained language model for further processing for determining the recommendation information.

In a further method step, a similarity algorithm is applied to the patient feature vector and the different protocol feature vectors. By means of the similarity algorithm, a measure of a similarity between the patient feature vector and the individual protocol feature vectors is determined, and hence a measure of a match between the patient feature vector and the individual protocol feature vectors is determined. Such similarity algorithms are known from the prior art. For example, the similarity algorithm can comprise a cosine similarity, for which an angle is ascertained between two vectors, in particular the patient feature vector and a protocol feature vector. A similarity value of 1 or close to 1 covers an angle of 0° or close to 0° between the two vectors, in particular the patient feature vector and a protocol feature vector. This value indicates that the two vectors, in particular the patient feature vector and a protocol feature vector, are almost identical. A similarity value of 0 or close to 0 indicates that the two vectors, in particular the patient feature vector and a protocol feature vector, have almost no match. Other known examples of a similarity algorithm are Euclidean distance (L2 norm) or dot product similarity. In addition, further similarity algorithms considered useful by a person skilled in the art are also readily conceivable.

The applying of the similarity algorithm may be performed by means of the computer, wherein the computer, in order to determine the recommendation information, accesses the patient feature vector provided by the trained LLM and the different protocol feature vectors provided by the trained LLM. The recommendation information comprises at least one magnetic resonance protocol, wherein the protocol feature vector of the at least one magnetic resonance protocol in the recommendation information has a maximum match with the patient feature vector. The maximum match comprises the greatest match between the different protocol feature vectors and the patient feature vector.

The recommendation information may include only that magnetic resonance protocol for which the protocol feature vector has the highest match with the patient feature vector. Alternatively, or additionally, the recommendation information can also include a ranking list, in which are listed the individual magnetic resonance protocols in the order in which the protocol feature vectors of the individual magnetic resonance protocols match the patient feature vector.

In addition, the recommendation information is provided. The providing of the recommendation information may be performed by means of a provision interface and/or an output interface of the computer. The recommendation information may be provided for output, in particular visual output, to the medical operating personnel. The output of the recommendation information may be performed by means of an output unit, in particular a visual output unit, such as a monitor and/or a display, for example, of a user interface.

The disclosure can advantageously provide a rapid recommendation of at least one magnetic resonance protocol to medical operating personnel during planning of the magnetic resonance examination. In particular, the disclosure can ensure that all the information available about the patient can be taken into account in determining the recommendation information and hence also when planning the magnetic resonance examination. A further advantage is that the recommendation information is based on a neutral analysis of the different magnetic resonance protocols in order to determine the best match between the examination requirements and the individual magnetic resonance protocols. In particular, this also allows a neutral assessment and recommendation of magnetic resonance protocols that are rarely or never considered in the case of manual selection because of a preference of the medical operating personnel and/or because of a complex workflow.

The disclosure can provide a method that considers information from different sources and/or information in different formats in determining the recommendation information. In addition, all current magnetic resonance protocols and also new protocol variants of a magnetic resonance protocol are always considered in determining the recommendation information. A further advantage of the disclosure is that a trained LLM provides an instrument that can be used advantageously to achieve high speed and/or high efficiency for providing recommendation information for the magnetic resonance examination.

In an advantageous development of the method according to the disclosure, it can be provided that the at least one item of examination-relevant patient information comprises at least one of the following items of information:
data and/or information on a previous examination of the patient and/or the progression of a disease of the patient up to now;
data and/or information that was created by medical operating personnel in preparation for the upcoming magnetic resonance examination of the patient; and/or
notes and/or comments that were created about the patient by medical operating personnel.

The examination-relevant patient information may comprise digital patient information stored in a database, in particular in a patient database and/or RIS database. In particular, the examination-relevant patient information comprises all the patient information held in a database or even in a plurality of databases.

The previous examination of the patient can be, for example, a preliminary examination for the upcoming magnetic resonance examination. It is also possible that the previous examination comprises an examination relating to a clinical and/or diagnostic issue that differs from the clinical and/or diagnostic issue of the upcoming magnetic resonance examination.

The notes and/or comments that were created about the patient by medical personnel, for instance a doctor and/or a radiologist, can comprise, for example, a medical report and/or further comments. In addition, the examination-relevant patient information can also comprise further patient data, such as an age of the patient, a size and/or weight of the patient, etc., for example.

This embodiment of the disclosure has the advantage that all the available information and/or data on the patient that is stored in a database is taken into account in determining the recommendation information for the upcoming magnetic resonance examination. For example, a patient may have a medical condition and/or injury that allows only a short stay in a magnetic resonance apparatus but is unrelated to the reason for the upcoming magnetic resonance examination. By taking into account all the information on the patient, the recommendation information can thus comprise a magnetic resonance protocol that has only a short measurement time.

In an advantageous development of the method according to the disclosure, it can be provided that the at least one item of examination-relevant operator information comprises an owner-dependent and/or local instruction and/or regulation for the magnetic resonance examination. The owner-dependent and/or local instruction and/or regulation can comprise, for example, a hospital-internal instruction for performing certain magnetic resonance examinations. In addition, the owner-dependent and/or local instruction and/or regulation can also comprise country-dependent regulations for performing certain magnetic resonance examinations. The determining of the recommendation information can thereby advantageously take into account owner-dependent information and/or local operator information.

In an advantageous development of the method according to the disclosure, it can be provided that the at least one item of examination-relevant patient information and/or the at least one item of examination-relevant operator information comprises free text. The free text does not include any conditions on a defined data structure and/or a defined text structure and/or a defined text format for the existing text. It is also possible that the free text may contain typing errors and/or spelling mistakes and/or grammatical errors. This can enable simple and rapid providing of the at least one item of examination-relevant patient information and/or the at least one item of examination-relevant operator information. In particular, it is thereby advantageously possible to dispense with time-consuming formatting of the at least one item of examination-relevant patient information and/or the at least one item of examination-relevant operator information before providing the at least one item of examination-relevant patient information and/or the at least one item of examination-relevant operator information for determining the recommendation information.

In an advantageous development of the method according to the disclosure, it can be provided that the providing of the at least one item of examination-relevant patient information and/or the at least one item of examination-relevant operator information comprises generating a text string from the at least one item of examination-relevant patient information and/or the at least one item of examination-relevant operator information. In particular, the generating of a text string comprises generating a single text string for all available examination-relevant patient information and available examination-relevant operator information. The text string may comprise a character string and/or a string of text characters. This embodiment of the disclosure enables simple and direct information generation by a trained language model, in particular an LLM.

In an advantageous development of the method according to the disclosure, it can be provided that the providing of protocol information on different magnetic resonance protocols from the totality of available magnetic resonance protocols comprises restricting protocol information to different magnetic resonance protocols in a protocol group. The totality of the available magnetic resonance protocols may comprise a plurality of protocol groups. Each of the plurality of protocol groups may be restricted to magnetic resonance protocols of a defined examination region. The defined examination region can comprise, for example, the head or the knee or the heart, etc. The providing of protocol information on different magnetic resonance protocols can comprise providing protocol information about all the magnetic resonance protocols assigned to a protocol group. The determining of the recommendation information can thereby advantageously take into account all magnetic resonance protocols that are relevant to the upcoming magnetic resonance examination.

The selection of a protocol group can be made automatically by the computer, for example on the basis of the examination-relevant patient information. In addition, the selection of a protocol group can also be made by medical operating personnel.

In an advantageous development of the method according to the disclosure, it can be provided that the providing of protocol information on different magnetic resonance protocols comprises generating a text string from each item of protocol information of a magnetic resonance protocol. In other words, a text string is generated for each magnetic resonance protocol of the different magnetic resonance protocols on the basis of the associated protocol information, resulting in a generated text string for each of the different magnetic resonance protocols. This embodiment of the disclosure enables simple and direct information generation by a trained language model, in particular an LLM.

In an advantageous development of the method according to the disclosure, it can be provided that the trained language model, in particular the LLM, comprises an encoder network, which is based on a neural network and is configured to encode the text strings. The encoder network may be trained to encode information from the respective text strings of the at least one item of examination-relevant patient information and/or of the at least one item of examination-relevant operator information and/or of the protocol information. This encoding is used to determine the patient feature vector and/or the protocol vectors. The patient feature vector comprises the encoding of the at least one item of examination-relevant patient information and/or the at least one item of examination-relevant operator information. The protocol feature vectors comprise the encoding of the protocol information. The encoding enables a simple and fast comparison between the patient feature vector and the different protocol feature vectors.

In an advantageous development of the method according to the disclosure, it can be provided that the recommendation information comprises a plurality of magnetic resonance protocols, with the plurality of magnetic resonance protocols sorted in terms of a match between the patient feature vector and the protocol feature vector of the magnetic resonance protocol concerned. The plurality of magnetic resonance protocols may be sorted such that the magnetic resonance protocols that have the highest match with the patient feature vector are listed first. A user, in particular the medical operating personnel, can thereby obtain recommendation information that contains an advantageous summary of the plurality of magnetic resonance protocols and their match with the requirement for the magnetic resonance examination on the patient. This can advantageously assist the medical operating personnel in selecting a magnetic resonance protocol for the upcoming magnetic resonance examination.

In addition, the recommendation information can comprise a value of a match between the patient feature vector and the protocol feature vectors for the plurality of magnetic resonance protocols. This additionally enables the medical operating personnel to select a suitable magnetic resonance protocol on the basis of the value of the match of the particular magnetic resonance protocol and, for instance, personal experience in using and/or executing the magnetic resonance protocol.

In an advantageous development of the method according to the disclosure, it can be provided that in a further method step, the provided recommendation information is output to a user. The output may be performed by means of an output unit, in particular a visual output unit, such as a monitor and/or a display. The output unit may be comprised by a user interface. The user interface can be comprised by the magnetic resonance apparatus. In addition, it is also conceivable that the user interface is comprised by a planning unit, which is designed for planning the magnetic resonance examination, or by a computer. This can achieve advantageous communication of the recommendation information to the user.

In an advantageous development of the method according to the disclosure, it can be provided that the output of the recommendation information comprises a default setting for a protocol selection for planning the magnetic resonance examination of the patient. This can make it easy to adopt the recommendation information for the magnetic resonance examination of the patient. For adopting the recommendation, the medical operating personnel can simply confirm the pre-selection, and the recommended magnetic resonance protocol is selected. In addition, this also gives the medical operating personnel the option to decline the recommendation and to make another protocol selection.

In an advantageous development of the method according to the disclosure, it can be provided that the language model, in particular the LLM, is retrained on the basis of the determined recommendation information. The language model, in particular the LLM, can also be adapted in this way. For example, by adapting the language model, in particular the LLM, magnetic resonance protocols that have a high match with defined examination requirements have a higher match value when the method is performed again. In contrast, magnetic resonance protocols that have a low match with defined examination requirements have a lower match value when the method is performed again.

This also enables user feedback on determined recommendation information to be incorporated in the trained language model, in particular the trained LLM. For example, medical operating personnel can thereby indicate whether the recommendation information was helpful or whether the recommended magnetic resonance protocols have actually contributed to clarifying the clinical and/or diagnostic issue.

The disclosure is also based on a computer for regulating and/or controlling a method for providing recommendation information for at least one magnetic resonance protocol for a magnetic resonance examination on a patient, wherein the computer comprises:
an input interface, which is configured to access at least one database, in which are stored the at least one item of examination-relevant patient information and/or the at least one item of examination-relevant operator information and/or the protocol information on different magnetic resonance protocols, in order to receive the at least one item of examination-relevant patient information and/or the at least one item of examination-relevant operator information and/or the protocol information;
an analysis module (analyzer), which is configured to provide by means of a trained language model a patient feature vector on the basis of the at least one item of examination-relevant patient information and/or the at least one item of examination-relevant operator information, and to provide by means of the trained language model a protocol feature vector for the protocol information of each magnetic resonance protocol, wherein the analysis module is additionally configured to determine, by means of a similarity algorithm, recommendation information on the basis of the patient feature vector and the protocol feature vectors; and
an output interface, which is configured to provide the recommendation information.

The computer can have a processor, which is comprised by the analysis module. The computer can also be a central computer and/or an edge device. The computer can be configured to execute the method according to the method aspect. Alternatively or additionally, the computer can comprise any feature disclosed in the context of the method according to the method aspect.

The computer can be realized as a data processing system or as part of a data processing system. Such a data processing system can comprise, for example, a cloud computing system, a computer network, a computer, a tablet computer, a smartphone, and/or the like. The computer can consist of hardware and/or software. The hardware can comprise, for example, one or more processors, one or more memories, and combinations thereof. The one or more memories can store instructions for executing the method steps according to the disclosure. The hardware can be configured by the software and/or operated by the software. In general, all the units, sub-units or modules can be understood to be in data communication with each other, at least temporarily, for instance via a network connection or suitable interfaces. Consequently, individual units can also be located remotely from each other.

The computer may include an interface unit, which may include the input interface and the output interface. The interface unit can comprise an interface for data communication with a local server or a central web server via an Internet connection for receiving data and/or information. In addition, the interface unit can be adapted to communicate with one or more users of the system, for instance by indicating to the user the result of the processing by the computer (for instance in a graphical user interface) or by enabling the user to adapt parameters for the data processing or visualization. In other words, the interface unit can comprise a user interface.

The advantages of the computer according to the disclosure are essentially the same as the advantages detailed above of the method according to the disclosure for providing recommendation information for at least one magnetic resonance protocol for a magnetic resonance examination. Features, advantages or alternative embodiments mentioned in this connection can be applied likewise to the other claimed subject matter, and vice versa.

In addition, the disclosure is based on a system for providing a recommendation of at least one magnetic resonance protocol for a magnetic resonance examination on a patient, comprising:
a computer, which is configured to regulate and/or control a method for providing recommendation information for at least one magnetic resonance protocol for a magnetic resonance examination on a patient;
at least one digital database in which is stored the at least one item of examination-relevant patient information and/or the at least one item of examination-relevant operator information and/or the protocol information.

The system may comprise two or more digital databases for storing the at least one item of examination-relevant patient information, the examination-relevant operator information, and the patient information. A first digital database of the system, for example a digital patient database or an RIS database, is configured to store the examination-relevant patient information. The examination-relevant operator information may be stored in a second digital database of the system. The protocol information may be stored in a further, third digital database. In addition, it can also be the case that the examination-relevant operator information and the protocol information are stored in a shared digital database.

The advantages of the system according to the disclosure are essentially the same as the advantages detailed above of the method according to the disclosure for providing recommendation information for at least one magnetic resonance protocol for a magnetic resonance examination. Features, advantages or alternative embodiments mentioned in this connection can be applied likewise to the other claimed subject matter, and vice versa.

According to a further aspect of the disclosure, a computer program product is provided with program elements which cause a computer to perform the steps of the method for providing recommendation information for at least one magnetic resonance protocol for a magnetic resonance examination on a patient according to the method aspect when the program elements are loaded into a memory of the computer.

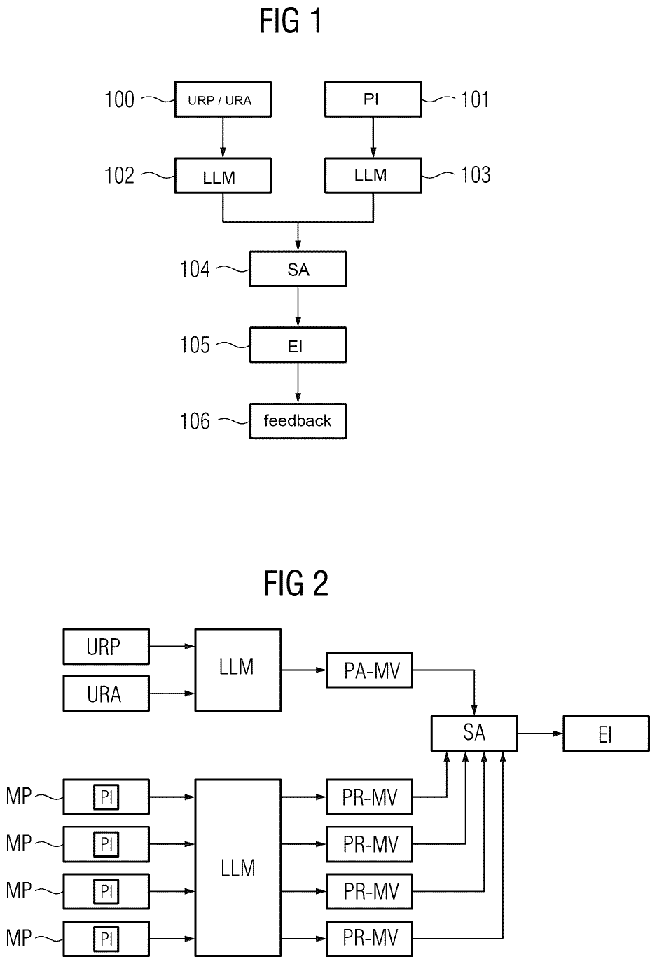

FIGS. 1 and 2 show a method according to the disclosure for providing recommendation information EI for at least one magnetic resonance protocol MP for a magnetic resonance examination on a patient. The method is controlled by a computer 10.

At least one item of examination-relevant patient information URP and/or at least one item of examination-relevant operator information URA is provided in a first method step 100. The providing is performed by a provision unit. The provision unit may be comprised by the computer 10, in particular by an input interface 11 of the computer 10. Alternatively or additionally, the provision unit can also be comprised by a database in which is stored the at least one item of examination-relevant patient information URP and/or the at least one item of examination-relevant operator information URA.

In an exemplary embodiment, the examination-relevant patient information URP comprises all the available patient information. The examination-relevant patient information URP can comprise data and/or information from a previous examination of the patient and/or the progression of a disease of the patient up to now. Alternatively or additionally, the examination-relevant patient information URP can also comprise data and/or information that was created by medical operating personnel in preparation for the upcoming magnetic resonance examination of the patient. Alternatively or additionally, the examination-relevant patient information URP can also comprise notes and/or comments that were created about the patient by medical personnel. In addition, the examination-relevant patient information URP can also comprise further patient data, such as the age of the patient, the size and/or weight of the patient, etc.

The examination-relevant operator information URA can comprise examination-relevant information of an operator, where an operator can be a doctor, a hospital, a radiology center, etc. The examination-relevant operator information can comprise, for example, a local and/or owner-dependent regulation and/or information relating to the magnetic resonance examination. The owner-dependent and/or local instruction and/or regulation can comprise, for example, a hospital-internal instruction for performing certain magnetic resonance examinations. In addition, the owner-dependent and/or local instruction and/or regulation can also comprise country-dependent regulations for performing certain magnetic resonance examinations.

The at least one item of examination-relevant patient information URP and/or the at least one item of examination-relevant operator information URA are formulated and/or may be stored as free text. The providing of the at least one item of examination-relevant patient information URP and/or the at least one item of examination-relevant operator information URA can comprise also generating a text string from the at least one item of examination-relevant patient information URP and/or the at least one item of examination-relevant operator information URA.

In a further, second method step 101, protocol information PI on different magnetic resonance protocols MP from a totality of available magnetic resonance protocols MP is provided. The providing is performed by the input interface 11 of the computer 10, in particular by the provision unit. This second method step 101 can be executed at the same time as the first method step 100.

The protocol information PI for a magnetic resonance protocol MP can comprise, in the simplest case, just the name of the magnetic resonance protocol MP. In an exemplary embodiment, the protocol information PI for a magnetic resonance protocol MP comprises additional information on the magnetic resonance protocol MP, such as which magnetic resonance sequences are used in the magnetic resonance protocol MP and/or what properties this magnetic resonance protocol MP comprises, etc.

The providing of protocol information PI on different magnetic resonance protocols MP from the totality of available magnetic resonance protocols MP may comprise restricting protocol information PI to different magnetic resonance protocols MP in a protocol group. In an exemplary embodiment, the totality of the available magnetic resonance protocols MP is divided into different protocol groups, where each different protocol group is assigned to a defined body region and may comprise all the magnetic resonance protocols MP for this defined body region. The provided protocol information PI can hence be restricted to those magnetic resonance protocols MP that are designed for measurements on the relevant body region of the patient that is to be examined.

For example, for a clinical and/or diagnostic issue relating to the head region of the patient can be provided only magnetic resonance protocols MP that are assigned to the “head” protocol group and designed and/or developed for head examinations. In another example, if the patient has a knee problem, can be provided only magnetic resonance protocols MP that are assigned to the “knee” protocol group and designed and/or developed for knee examinations.

The selection of a protocol group can be made automatically by the computer 10, for example on the basis of the examination-relevant patient information URP. In addition, the selection of a protocol group can also be made by medical operating personnel.

The providing of the different items of protocol information PI additionally comprises generating a text string. This involves generating a dedicated text string for each item of provided protocol information PI of a magnetic resonance protocol MP.

In a further, third method step 102, a trained language model LLM is applied to the provided examination-relevant patient information URP and/or the provided examination-relevant operator information URA, in particular to the text string generated from the examination-relevant patient information URP and/or the examination-relevant operator information URA. The trained language model comprises a trained large language model LLM. The LLM is used to determine and provide from the text string a patient feature vector PA-MV. The LLM comprises an encoder network, which is based on a neural network and is configured to encode the text string and provides this encoding in the patient feature vector PA-MV.

In a fourth method step 103, the LLM is applied to the provided protocol information PI of the different magnetic resonance protocols MP, in particular to the text strings generated from the protocol information PI of the different magnetic resonance protocols MP. The LLM is used to determine and provide from the text string a protocol feature vector PR-MV for each of the different magnetic resonance protocols MP. In particular, the LLM is used to determine an encoding of the text strings and to provide this encoding in the protocol feature vector PR-MV. The third method step 102 and the fourth method step 103 may be executed at the same time.

In a further, fifth method step 104, a similarity algorithm SA is applied to the patient feature vector PA-MV and the different protocol feature vectors PR-MV. The similarity algorithm SA is used to determine a measure of a similarity between the patient feature vector PA-MV and the individual protocol feature vectors PR-MV, and hence to determine a match between the patient feature vector PA-MV and the individual protocol feature vectors PR-MV. In one or more aspects, a conventional similarity algorithms SA may be used. For example, the similarity algorithm SA can comprise a cosine similarity algorithm or Euclidean distance (L2 norm) or dot product similarity, etc.

The similarity algorithm SA is used to generate and/or determine recommendation information EI. The recommendation information EI comprises at least one magnetic resonance protocol MP for which the protocol feature vector PR-MV has a maximum match with the patient feature vector PA-MV. The similarity algorithm SA is used to determine a similarity with the patient feature vector PA-MV for all the protocol feature vectors PR-MV. The protocol feature vector PR-MV of a magnetic resonance protocol MP that has the highest match with the patient feature vector PA-MV has a maximum match.

The recommendation information EI may comprise a plurality of magnetic resonance protocols MP, with the plurality of magnetic resonance protocols MP sorted in terms of a match between the patient feature vector PA-MV and the protocol feature vector PR-MV of the magnetic resonance protocol concerned MP. In particular, the plurality of magnetic resonance protocols MP are sorted such that the magnetic resonance protocols MP that have the highest match with the patient feature vector PA-MV are listed first.

In a further, sixth method step 105, the recommendation information EI is provided. The providing of the recommendation information EI may be performed by means of an output interface 12 of the computer 10.

Once the recommendation information EI has been provided, then in a further, subsequent seventh method step 106, the provided recommendation information EI can be output to the user. The output may be performed by means of a visual output unit 13, for example a monitor and/or a display. The output unit 13 may be comprised by the computer 10, such as by a user interface 14 of the computer 10. The output may just comprise the recommendation information EI, for example the one recommended magnetic resonance protocol MP or a list with the recommended magnetic resonance protocols MP. In addition, the output of the recommendation information EI can also comprise a default setting for a protocol selection for planning the magnetic resonance examination of the patient. For adopting the recommendation, the medical operating personnel can simply confirm the pre-selection, and the recommended magnetic resonance protocol MP is selected. Alternatively, the user, in particular the medical operating personnel, can decline the pre-selection if they do not agree with the protocol selection.

The recommendation information EI and/or feedback from the medical operating personnel on the recommendation information EI can also be supplied again, in the method steps 105 and 106, to the computer 10, in particular to the LLM and/or the similarity algorithm SA, in order to improve the determining of the patient feature vector PA-MV and/or the protocol feature vectors PR-MV and/or the recommendation information EI.

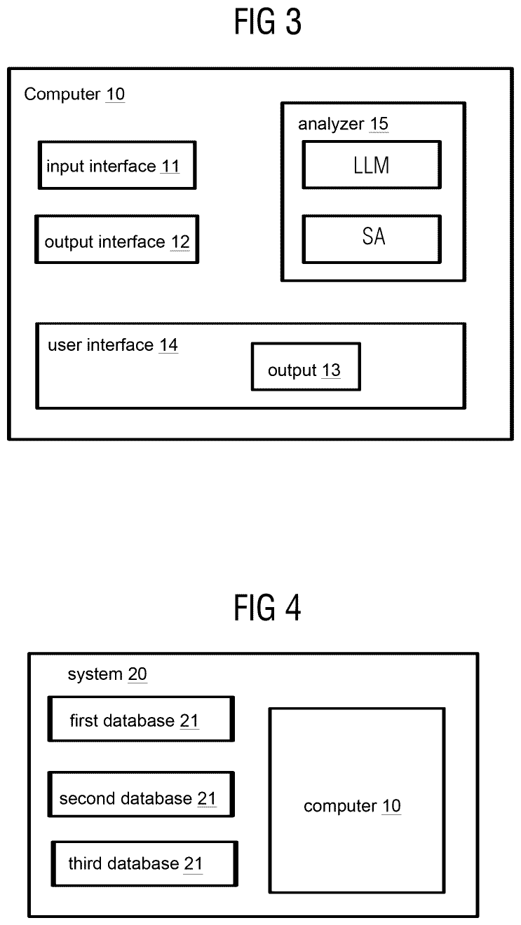

FIG. 3 shows the computer 10 in greater detail. The computer 10 may be configured to control the method for providing recommendation information EI for at least one magnetic resonance protocol MP for a magnetic resonance examination on a patient. For this purpose, the computer 10 has an input interface 11, an analysis module (analyzer) 15, and an output interface 12. The computer 10 may include processing circuitry configured to perform one or more functions and/or operations of the computer 10. Additionally, or alternatively, one or more components of the computer 10 may include processing circuitry that is configured to perform one or more respective functions of the component(s).

The input interface 11 may be configured to access at least one database 21 (e.g., a digital database 21) or a plurality of databases 21, such as digital databases 21. In these databases 21, the examination-relevant patient information URP and/or the examination-relevant operator information URA and the protocol information PI on the individual magnetic resonance protocols MP are stored. For example, a first database 21 comprises a patient database and/or an RIS database, in which the examination-relevant patient information URP is stored. In an exemplary embodiment, all the examination-relevant patient information URP is stored in the first database 21. The examination-relevant operator information URA can be stored in a second database 21, for example. In a further, third database 21 it is possible to store, for example, the protocol information PI on the different magnetic resonance protocols MP. The examination-relevant patient information URP and/or the examination-relevant operator information URA and/or the protocol information PI on the different magnetic resonance protocols MP can be received by means of the input interface 11.

The analysis module (analyzer) 15 may comprise the trained LLM and the similarity algorithm SA. The computer 10 can have a processor, which may be comprised by the analysis module 15. The analysis module 15 may be configured to provide, by means of the trained LLM, a patient feature vector PA-MV on the basis of the examination-relevant patient information URP and/or the at least one item of examination-relevant operator information URA and to provide a protocol feature vector PR-MV for the protocol information PI of each magnetic resonance protocol MP. In addition, the analysis module 15 may also be configured to determine, by means of the similarity algorithm SA, recommendation information EI on the basis of the patient feature vector PA-MV and the protocol feature vectors PR-MV.

The output interface 12 of the computer 10 may be configured to provide the recommendation information EI.

The computer 10 may be configured to control and/or execute the method for providing recommendation information EI for at least one magnetic resonance protocol MP for a magnetic resonance examination. The computer 10 may include a computer program product with program elements. The program elements may be configured to cause the computer 10 to execute the steps of the method for providing recommendation information EI for at least one magnetic resonance protocol MP for a magnetic resonance examination when the program elements are loaded into a memory of the computer 10.

FIG. 4 shows a system 20 for providing a recommendation for at least one magnetic resonance protocol MP for a magnetic resonance examination on a patient. The system 20 comprises the computer 10, which was already described in detail in the description relating to FIG. 3. In addition, the system 20 has at least one digital database 21. In the present exemplary embodiment, the system 20 has three digital databases 21. In a first digital database 21, for instance in a patient database and/or an RIS database, is stored the examination-relevant patient information URP. Stored in a second digital database 21 is the examination-relevant operator information URA. Stored in a third digital database 21 is the protocol information PI on the different magnetic resonance protocols MP. The system 20 can further comprise an output unit for output of the recommendation information EI. The system 20 may include processing circuitry configured to perform one or more functions and/or operations of the system 20. Additionally, or alternatively, one or more components of the system 20 may include processing circuitry that is configured to perform one or more respective functions of the component(s).

Although the disclosure has been illustrated and described in detail using the exemplary embodiment, the disclosure is not limited by the disclosed examples, and a person skilled in the art can derive other variations therefrom without departing from the scope of protection of the disclosure.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

The various components described herein may be referred to as “modules,” “units,” or “devices.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such modules, units, or devices, as applicable and relevant, may alternatively be referred to herein as “circuitry,” “controllers,” “processors,” or “processing circuitry,” or alternatively as noted herein.

For the purposes of this discussion, the term “processing circuitry” shall be understood to be circuit(s) or processor(s), or a combination thereof. A circuit includes an analog circuit, a digital circuit, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A computer-implemented method for providing recommendation information for at least one magnetic resonance protocol for a magnetic resonance examination on a patient, the method comprising:
providing at least one item of examination-relevant patient information and/or at least one item of examination-relevant operator information;
providing protocol information on different magnetic resonance protocols from a totality of available magnetic resonance protocols;
determining, by applying a trained language model to the at least one item of examination-relevant patient information and/or the at least one item of examination-relevant operator information, a patient feature vector;
determining, by applying the trained language model to protocol information of the different magnetic resonance protocols, a protocol feature vector for each magnetic resonance protocol of the different magnetic resonance protocols;
applying a similarity algorithm to the patient feature vector and the different protocol feature vectors to determine recommendation information comprising at least one magnetic resonance protocol, wherein the protocol feature vector of the at least one magnetic resonance protocol in the recommendation information has a maximum match with the patient feature vector; and
providing the recommendation information to facilitate the magnetic resonance examination.

2. The method as claimed in claim 1, wherein the at least one item of examination-relevant patient information comprises at least one of:
data and/or information on a previous examination of the patient and/or a current progression of a disease of the patient;
data and/or information that was created by medical personnel in preparation for the upcoming magnetic resonance examination of the patient; and/or
notes and/or comments that were created about the patient by medical personnel.

3. The method as claimed in claim 1, wherein the at least one item of examination-relevant operator information comprises an owner-dependent and/or local instruction, and/or regulation for the magnetic resonance examination.

4. The method as claimed in claim 1, wherein the at least one item of examination-relevant patient information and/or the at least one item of examination-relevant operator information comprises free text.

5. The method as claimed in claim 1, wherein the providing of the at least one item of examination-relevant patient information and/or the at least one item of examination-relevant operator information comprises generating a text string from the at least one item of examination-relevant patient information and/or the at least one item of examination-relevant operator information.

6. The method as claimed in claim 1, wherein the providing of protocol information on different magnetic resonance protocols from the totality of available magnetic resonance protocols comprises restricting protocol information to different magnetic resonance protocols in a protocol group.

7. The method as claimed in claim 1, wherein the providing of protocol information on different magnetic resonance protocols comprises generating a text string from each item of protocol information of a magnetic resonance protocol.

8. The method as claimed in claim 5, wherein the trained language model comprises an encoder network, which is based on a neural network and is configured to encode the text strings.

9. The method as claimed in claim 1, wherein the recommendation information comprises a plurality of magnetic resonance protocols, with the plurality of magnetic resonance protocols sorted in terms of a match between the patient feature vector and the protocol feature vector of the magnetic resonance protocol concerned.

10. The method as claimed in claim 1, further comprising outputting the provided recommendation information to a user.

11. The method as claimed in claim 10, wherein the output of the recommendation information comprises a default setting for a protocol selection for planning the magnetic resonance examination of the patient.

12. The method as claimed in claim 1, further comprising retraining the language model based on the determined recommendation information.

13. The method as claimed in claim 1, further comprising configuring, based on the recommendation information, a magnetic resonance apparatus with the at least one magnetic resonance protocol from the recommendation information.

14. The method as claimed in claim 1, further comprising executing the magnetic resonance examination on the patient using the at least one magnetic resonance protocol from the recommendation information.

15. The method as claimed in claim 1, wherein the trained language model comprises a large language model comprising an encoder network based on a neural network, wherein the encoder network is configured to encode the at least one item of examination-relevant patient information and/or the at least one item of examination-relevant operator information to determine the patient feature vector, and to encode the protocol information to determine the protocol feature vectors.

16. The method as claimed in claim 1, further comprising:
outputting the recommendation information as a default setting for a protocol selection; and
based on a confirmation associated with the default setting, selecting the at least one magnetic resonance protocol for the magnetic resonance examination.

17. One or more non-transitory media storing instructions that, when executed by one or more processors, cause the one or more processors to perform the method of claim 1.

18. A computer for providing recommendation information for at least one magnetic resonance protocol for a magnetic resonance examination on a patient, the computer comprising:
an input interface configured to access at least one database storing the at least one item of examination-relevant patient information, at least one item of examination-relevant operator information, and/or the protocol information on different magnetic resonance protocols, to receive the at least one item of examination-relevant patient information, the at least one item of examination-relevant operator information, and/or the protocol information on different magnetic resonance protocols;
an analyzer configured to:
provide, using a trained language model, a patient feature vector based on the at least one item of examination-relevant patient information and/or the at least one item of examination-relevant operator information,
provide, using the trained language model, a protocol feature vector for the protocol information of each magnetic resonance protocol, and
determine, using a similarity algorithm, recommendation information based on the patient feature vector and the protocol feature vectors; and
an output interface configured to provide the recommendation information to facilitate the magnetic resonance examination.

19. A system for providing a recommendation for at least one magnetic resonance protocol for a magnetic resonance examination on a patient, comprising:
the computer of claim 18, and
at least one digital database in which is stored the at least one item of examination-relevant patient information and/or the at least one item of examination-relevant operator information and/or the protocol information.
